(19) United States
(12) Patent Application Publication
(10) Pub. No.: US 2016/0022155 A1
(43) Pub. Date: Jan. 28, 2016

(54) Evaluating the Cardiac Engine

(71) Applicant: Stephanie LITTELL, Lexington, MA (US)

(72) Inventor: Stephanie LITTELL, Lexington, MA (US)

(21) Appl. No.: 14/774,462

(22) PCT Filed: Mar. 14, 2014
(86) PCT No.: PCT/US14/27999

§ 371 Date: Sep. 10, 2015

Related U.S. Application Data
(60) Provisional application No. 61/799,327, filed on Mar. 15, 2013.

(51) Int. Cl. A61B 5/02; A61B 5/145; A61B 5/00; A61B 5/107

(52) U.S. Cl. CPC A61B 5/02028; A61B 5/1073; A61B 5/1072; A61B 5/14542; A61B 5/742; A61B 5/0059; A61B 5/0022; A61B 2562/04

(57) ABSTRACT

The present invention relates to computer-based methods for evaluating an individual's cardiovascular system and computer systems for use in such methods. The computer system recognizes a portion of the individual's cardiovascular system though an imaging device input, then assesses the cardiac power, energy and efficiency during the cardiac cycle. The computer system evaluates circulatory motion and properties of circulatory fluid, and further optimizes the cardiovascular analysis through suggested repositioning of the imaging device.

### RELATED APPLICATIONS

This application claims the benefit of U.S. Provisional Application No. 61/799,327, filed on Mar. 15, 2013. The entire teachings of the above application are incorporated herein by reference.

### BACKGROUND OF THE INVENTION

Typically, measures of cardiac health focus on the heart tissue, or on functional metrics such as ventricular ejection fraction, blood pressure, ventricular volume, stroke volumes, and cardiac output. Because of the complexity of the heart, it has been difficult to develop a methodology sufficient for a detailed and informative assessment of the cardiac system.

Cardiovascular disease, which encompasses any disease of the cardiovascular system, is one of the leading causes of death in the United States and world-wide. Assessing cardiac function provides a measure of cardiac health and thus provides insight into whether an individual is suffering from cardiovascular disease or is at risk for suffering from cardiovascular disease. In order to assess cardiac function, non-invasive measurements, for example resting heart rate and blood pressure, can be taken. Certain other measurements of heart function require invasive techniques and measure, for example, stroke volume, ejection fraction, cardiac output, and chamber pressures in the left and right atrium and ventricle.

Normal cardiac function may be characterized as generative output energy by the heart. By studying an individual's generative output energy, cardiovascular disease can be better studied and its progression can be tracked over time. Digital tools enable understanding of generative output energy and cardiac output energy.

Known methods of cardiovascular evaluation are limited in that the methods do not account for the production of fluid uptake properties, for example nutrient levels or fluid forces. Furthermore, known methods do not account for the ways in which cardiac geometry affects energy production and delivery, the role of gravity in cardiac velocity trajectories, the effects of geometrical junctures on oxygenated fluid delivery, or the role of stimulation around the chamber perimeter. There remains a need to develop methods for evaluating a cardiovascular system that address the limitations in the known art, and furthermore that are non-invasive, reliable, less expensive, and that address these additional aspects of health assessment.

### SUMMARY OF THE INVENTION

Embodiments of the present invention address the foregoing problems and shortcomings in the art. In particular, the present invention provides a computer system and method for evaluating an individual's heart and vasculature as a cardiac engine system.

In one embodiment, the invention relates to a computer system for evaluating an individual's cardiac system. The computer system comprises a recognition module configured to recognize a portion of the individual's cardiovascular system, a geometry module coupled to the recognition module and configured to assess a dimensional geometry, physiology, or a combination thereof of the recognized portion of the individual's cardiovascular system at a cardiac cycle time t, an interval selection module configured to select a cardiac cycle interval, and an interval evaluation module responsive to the geometry module's assessment of the recognized portion of the individual's cardiovascular system and configured to compute current, cardiac power, cardiac energy, efficiency, or a combination thereof of the individual's cardiovascular system at the selected cardiac cycle interval, cardiac cycle time t, or a combination thereof, wherein the interval evaluation module indicates an evaluation of cardiac energy and efficiency.

In certain embodiments, the dimensional geometry that is assessed is area, volume, length, relative orientation, or a combination thereof of the recognized portion of the individual's cardiovascular system at the cardiac cycle time t.

The invention also provides for the computer system to further comprise a circulatory fluid property module configured to determine a permeability, a deformability, an oxygenation, or a combination thereof of a circulatory fluid.

The invention also provides for the computer system to further comprise a pulsatility module configured to determine a rotational velocity, a translational velocity, a volume, or a combination thereof by gauging one or more geometries and one or more velocities of a pulse wavefront and by gauging deformation of blood vessels.

The invention also provides for the computer system to further comprise a circulatory effect module configured to display a circulatory motion in one or more regions of the individual's cardiovascular system.

The invention also provides for the computer system to further comprise a boosting module configured to determine changes in vascular energy delivery.

The invention also provides for the computer system to further comprise a sensor module configured to connect one or more sensors.

The invention also provides for the computer system to further comprise a device module configured to connect one or more imaging device. The imaging device, in certain embodiments, is a camera.

The invention also provides for the computer system to further comprise a recommendation module configured to suggest repositioning of one or more sensors or one or more imaging devices.

The evaluation of cardiac energy and efficiency is an evaluation of incoming energy, output energy, kinetic energy, translational kinetic energy, rotational kinetic energy, or potential energy, at the selected cardiac cycle interval or the cardiac cycle time t.

In another embodiment, the computer system further comprises a flux state module configured to evaluate inductive λ of the individual's cardiovascular system at the cardiac cycle time t and a magnetic permeability module configured to compute μ at the cardiac cycle time t.

The present invention also provides that the computer system further comprises a cross-chambers module configured to determine differences in function between heart chambers.

The present invention also relates to methods of evaluating an individual's cardiovascular system. The method comprises (a) recognizing a portion of an individual's cardiovascular system, (b) assessing a dimensional geometry, physiology, or a combination thereof of the recognized portion of the individual's cardiac structure at a cardiac cycle time t, (c) selecting a cardiac cycle interval, and (d) computing current, cardiac power, cardiac energy, efficiency, or a combination thereof at the selected cardiac cycle interval, cardiac cycle time t or a combination thereof to generate an evaluation of the individual's cardiovascular system, wherein the evaluation is an assessment of cardiac energy and efficiency.

In certain embodiments, the dimensional geometry is area, volume, length, relative orientation, or a combination thereof at the cardiac cycle time t.

In certain embodiments, the evaluation of cardiac energy and efficiency, as achieved by the method of the present invention, is an evaluation of incoming energy, output energy, kinetic energy, potential energy, translational kinetic energy or rotational kinetic energy at the selected cardiac cycle interval or the cardiac cycle time t.

The invention also provides for the method to further comprise evaluating inductive λ of the individual's cardiovascular system and computing μ at cardiac cycle time t.

In another embodiment, the method further comprises suggesting repositioning of an imaging device, producing analysis of the evaluation of cardiac energy and efficiency, and transmitting the individual's evaluation to a target address.

In certain embodiments, producing analysis of the evaluation of cardiac energy and efficiency comprises reporting of energy production. In some embodiments, the target address is a printer, an electronic mail recipient or a Cloud computing device, server and the like.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a flow diagram of one embodiment of the present invention 100 relating to a method for evaluating an individual's cardiovascular system.

FIG. 2 is a schematic view of a computer network environment in which embodiments of the present invention 100 may be deployed.

FIG. 3 is a block diagram of computer nodes or devices in the computer network of FIG. 2 embodying the present invention.

### DETAILED DESCRIPTION OF THE INVENTION

A description of example embodiments of the invention follows.

In addition to being a biological organ, the heart can be considered as an engine pump. Therefore, throughout the description of the present invention, the heart is alternately referred to as a “cardiac engine.” The more effectively the cardiac engine pumps blood through the circulatory system, also referred to as the vasculature, the more efficient the cardiac engine is. Indicators of cardiac health include cardiac output and efficiency of the cardiac engine.

In order to produce efficient fluidic output, a well-functioning heart must apply rapid torquing force to rotate fluid, and translational force to streamline blood out into blood vessels. These forces can be measured, directly at the left ventricle, or just as blood enters the aorta past the aortic valve. At that point, the heart has completed its powering cycle, and ejected blood relative to its own degree of effectiveness.

In certain instances, an abnormal cardiac engine output, cardiac energy, and cardiac efficiency is an indicator of cardiovascular problems related to underlying disease and pathology. For example, a low efficiency in pumping is often noted with pathologies such as valve regurgitation, cardiac hypertrophy, or cardiac atrophy.

As used herein, “cardiac output” refers to the amount of blood delivered by the heart per minute.

An essential function of the heart is to provide and circulate the blood to supply nutrients throughout an organism or individual. Blood must travel through the vasculature, and deliver nutrients to organ systems and tissues through diffusion, absorption, or active or passive cellular transport. Typical nutrients delivered by blood include gases, ions, micromolecules, macromolecules, sugars, proteins, fats, fatty acids, nucleic acids, and cells. A coiled or spiral configuration of blood flowing through the body's vessels increases the amount of nutrients delivered through the blood, relative to a uniform tube of blood flow (i.e., laminar flow), due to the surface exposure of new fluidic layers per longitudinal inch. The rate at which energy is delivered correlates to powering the body of an individual.

In order to deliver nutrients to downstream organs and tissues, a significant amount of energy must be repeatedly generated by the heart. The total energy produced by the heart during the cardiac cycle, referred to herein as “cardiac energy,” is a dynamic combination of potential and kinetic energies. In certain embodiments, cardiac energy includes one or more of incoming energy, output energy, kinetic energy, translational kinetic energy, rotational kinetic energy, or potential energy. Potential energy is created through fluidic compression (e.g., systole) and any effective counter-gravitational change. “Generative output energy” is the energy an individual generates with respect to the enclosed fluid. In certain embodiments, generative output energy does not match well with overall cardiac output energy due to poor red blood cell properties, poor electrical stimulation, cardiac timing or abnormal geometry. Cardiac output energy includes energy put out by the heart for delivery to the vasculature. In certain embodiments, cardiac output energy is referred to as output energy.

Accordingly, in one embodiment, the invention is a computer system for evaluating an individual's cardiovascular system. The computer system comprises a recognition module configured to recognize a portion of the individual's cardiovascular system, a geometry module coupled to the recognition module and configured to assess a dimensional geometry, physiology, or a combination thereof of the recognized portion of the individual's cardiovascular system at a cardiac cycle time t, an interval selection module configured to select a cardiac cycle interval, and an interval evaluation module responsive to the geometry module's assessment of the recognized portion of the individual's cardiovascular system and configured to compute current, cardiac power, cardiac energy, efficiency, or a combination thereof of the individual's cardiovascular system at the selected cardiac cycle interval, cardiac cycle time t, or a combination thereof, wherein the interval evaluation module indicates an evaluation of cardiac energy and efficiency. The dimensional geometry that is assessed comprises area, volume, length, and relative orientation at the cyclic time t. In certain embodiments, the area assessed in the assessment of dimensional geometry is, for example, the area of a cross section of a portion of a cardiovascular system, or the surface area of a portion of an organ system, for example, for example the surface area of an inner or outer perimeter.

The present invention provides a cardiovascular evaluation to individuals looking for an understanding of their cardiac engine efficiency. The invention enables an individual to easily and accurately measure their own cardiac engine output and efficiency, similar to current individual-operated devices, for example, such as those that are used to measure blood pressure frequently found in grocery stores and pharmacies.

An individual's cardiac output energy or generative output energy, as put forth by the cardiovascular evaluation, allows an individual to see where he or she falls within predefined ranges of normal values for a given population. Specifically, an understanding of cardiac output energy or generative output energy would be useful to individuals who feel well and do not exhibit symptoms of cardiovascular disease, but who, for example, want to learn how they compare to an average individual along a spectrum of cardiac fitness. Similarly, certain individuals, for example professional athletes, may wish to obtain an evaluation of their cardiovascular system in order to learn the level of cardiac “power” that they typically generate. An evaluation of an individual's cardiovascular system would also enable an individual to understand why they lack energy, why they are short of breath after minimal exertion, or why they can no longer perform activities and tasks as often or as long as they used to. The pediatric and geriatric populations would especially benefit from a simple study of cardiac output energy or generative output energy, for example, rather than studies that require more invasive techniques.

In particular embodiments, individuals desiring additional information and specifics regarding their cardiovascular system elect to receive a tailored and detailed cardiac engine profile. Analysis using an individual's profile, which includes gender, age, set prior visits, past medical history, race, environmental factors, genetic dispositions, furnishes this tailored analysis. Furthermore, exercise or stress tolerance, body position, and force vectors could be furnished.

The recognition module is configured to recognize a portion of the individual's cardiovascular system. An imaging device senses or images a portion of the individual's cardiovascular system, which is then recognized by the recognition module, by, for example, the morphology or physiology of the portion of the cardiovascular system. A geometry module is configured to assess the dimensional geometry, physiology, or both dimensional geometry and physiology of the portion of the cardiovascular system at a cardiac cycle time t. The geometry module assesses, for example, the muscle tissue of the cardiac chambers, or alternately the enclosed fluidic cavities, and measure and assess area, volume, or a combination thereof of imaged slices. As used herein, “cardiac cycle time t” is any moment or instance in time within the cardiac cycle. In an example embodiment, a moment occurring at any time within the cardiac cycle is selected as t=0 by a user. As the cardiac cycle progresses, t increases in value. When the cardiac cycle begins a repeat cycle, cardiac cycle time t resets at t=0. In certain embodiments, the measurements and computations completed by the computer system and in the methods of the present invention can occur at more than one moment t during the cardiac cycle. For example, cardiac power can be computed at moment t=x, and later in the cardiac cycle at t=x+y.

The interval selection module is configured to choose a cardiac cycle interval for analysis. As used herein, “cardiac cycle interval” refers to an interval of time within a cardiac cycle or spanning a portion of each of two cardiac cycles. Cardiac interval can be, for example, systole, diastole, or the period of time between mitral valve closing and mitral valve opening.

At the interval evaluation module, current, cardiac power, cardiac energy, efficiency, or a combination thereof are computed at the selected cardiac cycle interval, cardiac cycle time t, or a combination thereof. The computation completed by the interval evaluation module provides an evaluation of cardiac energy and efficiency. Such an evaluation includes an evaluation of incoming energy, output energy, total kinetic energy, translational kinetic energy, rotational kinetic energy, or potential energy.

In certain embodiments, the computer system further comprises a field estimator module configured to estimate an electrical field, a magnetic field, or a combination thereof during the selected cardiac cycle interval. Magnetic field, alternately represented by the capital letter B, is a measure of the density magnetic flux at each point in the area or material concerned.

In certain embodiments, the computer system described by the present invention further comprises a device module configured to connect one or more imaging devices. In certain other embodiments, the computer system further comprises a sensor module configured to connect one or more sensors. In yet a further embodiment, the computer system comprises both a device module and a sensor module.

In certain embodiments, the imaging device that is used with the device module in evaluating an individual's cardiovascular system is a transducer or probe. In certain other embodiments, the imaging device is a camera. In an example embodiment, the imaging device is an ultrasound device. In certain other embodiments the imaging device further comprises a screen display, a module for emailing or uploading data to a server or a computer connected to the Internet, or a printer. In other embodiments, the imaging device is any electronic device capable of imaging. An imaging device can be connected to, or be part of a cell phone, tablet, laptop computer, portable device, or handheld computer. In yet further embodiments, imaging data is acquired through an electronic device, such as a cell phone or tablet, with one or more sensors.

Examples of sensors that can be used in evaluating an individual's cardiovascular system include magnetic sensors, gyroscopes, accelerometers, electrocardiography sensors, electromyography sensors, cameras, RGB sensors, green light sensors, blue light sensors, red light sensors or combinations thereof, motion sensors, near infrared cameras, infrared cameras, thermal cameras, GPS, or Wi-Fi.

In certain embodiments, the computer system of the present invention further comprise a flux state module configured to evaluate inductive λ of the individual's cardiovascular system at cardiac cycle time t. In further embodiments, the computer system also further comprises a magnetic permeability module configured to compute μ at cardiac cycle time t.

In certain embodiments, the computer system of the present invention further comprises a cross-chambers module configured to determine differences in function between heart chambers or between sets of heart chambers. In example embodiments, the differences in function that are assessed by the cross-chambers module include any lack of electrical stimulation per cardiac cycle, differences in permeability in oxygenated versus deoxygenated blood, phase evaluation, and chamber geometry.

In certain embodiments, imaging is tuned with a recommendation module. A recommendation module suggests repositioning of one or more sensors or one or more imaging devices by, for example, fine angle modification, switching between sensors and camera view, moving to a new portion of the cardiovascular system for further assessment, enlarging a view, or adjusting image output, for example through increasing or decreasing gain, brightness, contrast or noise.

The present invention further provides a showcasing module for viewing a portion of the individual's cardiovascular system. In example embodiments, the showcasing module visually emphasizes, abstracts, creates a freeze frame of, or animates the fully or partially functioning cardiovascular system, subsystem, subregion, metrics, or visual abstraction, such that each might be switched onscreen via pointing, clicking or gesturing upon and “opening” into the next animated cardiovascular subsystem, subregion, metric or summary aspect, or rotated to obtain another animated view, or zoomed to drill down into further detail.

In some embodiments, the computer system of the present invention further comprises a pulsatility module configured to determine rotational and translational velocities and volume by gauging the shape and speed of an arterial pulse wavefront and the deformation of blood vessels during fluidic delivery. In certain embodiments, the pulsatility module works in conjunction with the showcase module such that cardiac energy and efficiency local to a portion of the cardiovascular system is animated or displayed.

In other embodiments, the computer system further comprises a circulatory fluid property module configured to determine a permeability, a deformability, or an oxygenation of a circulatory fluid, or a combination of such properties. A circulatory fluid is, for example, blood, lymph, or cerebral spinal fluid.

In certain embodiments, the computer system further comprises a circulatory effect module, which displays a circulatory motion in one or more regions of the individual's cardiovascular system. For example, the circulatory effect module showcases the individual's head, face, or limbs. The circulatory effect module enables viewing a portion of the individual's cardiovascular system, and thus circulatory effect can also be displayed by a showcase module. In example embodiments, a circulatory motion is shown as a fine motion due to arterial pulsation, capillary bed influx and efflux, venous return, or a combination thereof. In other example embodiments, the circulatory effect module displays circulatory motion utilizing color to indicate the level of oxygenation. Alternately, the circulatory effect module works in conjunction with a triggering module to trigger an alert if any uneven, abnormal, or insufficient distribution of blood, red blood cells, oxygen, hemoglobin or any other blood component is observed within the cardiovascular system. The computer system of the present invention is also used to identify and characterize dead tissue, for example electrically dead cardiac tissue. In an example embodiment, lack of fluidic motion along the wall of a heart chamber during cyclic stimulation is indicative of dead cardiac tissue.

In other embodiments, the computer system further comprises a boosting module configured to determine changes in vascular energy delivery. In certain embodiments, changes in vascular energy delivery indicate energy changes in oxygenated fluidic current. In an example embodiment, arterial branching causes changes in oxygenated fluidic current and therefore vascular energy delivery changes.

In certain embodiments, the evaluation of an individual's cardiac system can include the discovery and characterization of dead tissue via electrical stimulation. Dead tissue is alternately referred to herein as “electrically dead tissue.” In certain embodiments, the computer system of the present invention identifies electrically dead tissue via flux calculations, or via assessing the heart chamber boundary, for example an inner or outer perimeter of the chamber, during stimulation and tracking tangential fluidic velocities, and assessment of geometry and permeability of a portion of the cardiovascular system.

In certain embodiments, the computer system further comprises a voltage integrator module configured to track integrated voltage from an EKG lead at cardiac cycle time t.

In yet further embodiments, the computer system of the present invention further comprises a reporting module configured to produce analysis of a cardiac evaluation including at least the assessment of cardiac energy and efficiency. The reporting module reports energy production. In certain embodiments, the report including the evaluative assessment of cardiac energy and efficiency further comprises profiling information about the individual. Such profiling information includes, for example, gender, age, diet, height, weight, athletic level or ability, or other physical characteristics of the individual. The system also comprises a transmittal module configured to send the cardiac evaluation data to one or more target addresses, and a setup module for starting a new individual assessment or configuring application options. As used herein, “sending” the cardiac evaluation means electronically communicating or otherwise transmitting the cardiac evaluation. In example embodiments, a target address is a printer, an electronic mail recipient, for example the electronic mail address of the individual or the spouse of the individual, or a Cloud computing device. In other embodiments, the cardiac evaluation data is stored or held by the computer system until a user or operator inputs target address information.

In certain embodiments, the computer system described herein further comprises a body transition module to switch a screen display from an external body view to an internal body view, from an internal body view to an external body view or to a display wherein both internal and external views are displayed. In certain embodiments, multiple views are displayed via tiling. In an example embodiment, views of the individual's face, neck, chest, and optionally another body part or a whole body view are tiled in a display.

The computer system described herein also includes a user options module in certain embodiments, wherein the module enables the user to configure the computer system to meet user-specified parameters. For example, the user options module configures the system to trigger a self-alert when a user- or physician-specified target cardiac energy is achieved. In another example, the user options module assesses cardiovascular age, compare cardiac engine to designated celebrity type, designate buddies for an interactive health circle, search for similar engine profiles by other application-using individuals, for providing palette or icon options to allow color, texture, contrast, and/or brightening to be applied to cardiac components selectively or to all.

In certain embodiments, the individual whose cardiovascular system is being studied is a mammal. In more particular embodiments, the mammal is a human.

In another embodiment, the computer system further comprises a cardiac phase events module to identify or select cardiac cycle data by cardiac physiology gating events such as valve open and close, diastole, systole, pre-diastole, post-systole, cycle time range, cycle time, and EKG gating events including P wave, QRS complex and T wave.

The present invention also provides for a method of evaluating an individual's cardiovascular system. The methods comprise (a) recognizing a portion of an individual's cardiovascular system, (b) assessing dimensional geometry, physiology, or a combination thereof of the recognized portion of the individual's cardiovascular system at a cardiac cycle time t, (c) selecting a cardiac cycle interval, and (d) computing current, cardiac power, cardiac energy, efficiency, or a combination thereof at the selected cardiac cycle interval, cardiac cycle time t, or a combination thereof, wherein the evaluation is an assessment of cardiac energy and efficiency.

The methods described herein further comprise, in certain embodiments, evaluating inductive λ of the individual's cardiovascular system and computing μ at cardiac cycle time t. In alternate embodiments, the methods further comprise suggesting repositioning of an imaging device, producing analysis of the evaluation of cardiac energy and efficiency, transmitting the individual's evaluation to a target address, as outlined above, or a combination thereof.

In certain embodiments, the method further comprises logging the evaluation data, printing an assessment, and sending one or more confirmation notices, for example via electronic mail, with the summary and accompanying specifics of the completed evaluation.

In certain embodiments, the method includes comparing cardiac cycle phases to assess energy production, power production, current flow in blood, flux states, measured or imputed electrical curves, or to impute blood's observed magnetic permeability as sampled curve.

In alternate embodiments, the method includes using a magnetic flux state machine to assess induction. In another embodiment, the method includes integrating EKG curve readings or stock curve to impute towards flux magnitude.

In another embodiment, the method includes comparing left versus right atrial and ventricle chamber induction, flux λ, permeability μ, and energy produced.

In another embodiment, the method includes inferring cardiac structure, for example, if the cardiac structure is not currently visible in an image, from motion hemodynamic gradient lines.

Another embodiment of the present invention comprises producing one or more comparative reports of cardiac energy.

Out from the Cardiac Engine

Kinetic energy generated by the heart, results in ejection of blood from the aortic valve. Kinetic energy is translational kinetic energy (Eₜ), rotational kinetic energy (Eᵣ), or a combination thereof.

Each kinetic component is observed as a fluidic motion, represented as vector quantities in distance per unit time (t), with modern imaging modalities and techniques.

$$\begin{matrix}{E}_{t} = \frac{1}{2} {mv}^{2} & Equation 1 \\ {E}_{r} = \frac{1}{2} {mr}^{2} {v}^{2} & Equation 2\end{matrix}$$

Equations 1 and 2, above, are measured through computing mass and velocity estimates in an observed sample box, radial area, or perceived actual disk slices of volume at the aortic valve or left ventricular outflow tract.

In other words, imputing mass outflow non-invasively amounts to mass fluxing, at a presumed density, through pertinent volume slices in an effective cross-section normal to aortic pipe longitudinal axis. Imputing corresponding velocities non-invasively amounts to spatial vectorizing of sampled velocity flux gradients through all similar pertinent volume slices.

Any rotational energy component will contribute more to outflow energy than translational energy, due to the extra positive radius squared term (r²) multiplied. In other words, larger and more closely-spaced “coils” of rotational fluidic outflow will deliver more energy.

Inside the Cardiac Engine

Working backwards, some simplistic assumptions about the heart itself yields first-level estimates of energy produced during phases prior to ejection. Future energy estimates are compared for phased energy production.

For example, fluidic velocity vectors, starting with the largest-magnitude rotational gradient relative to mitral valve orifice orientation, and mass flux through the mitral valve are observed or estimated. Kinetic energy is computed accordingly, prior to valve closure. Comparing such intermediate kinetic energy to ejected kinetic energy yields an estimate of the difference of effective systole kinetic energy production minus any calculated mass retained.

Drilling further into systole itself to compare to the above “sandwich gating,” before and after isovolumetric contraction, utilizing the rotational kinetic energy Equation 2, a heart's rotational velocities are observed and computed from its sampled radii, shape, and/or area slices. This calculation assumes a rigid rotating body, roughly cylindrical in shape, an average human heart mass by age, gender, or a combination thereof, and a sampled rotational degree of torsion at the apex, middle, base, or combination thereof. The actual measured size of various areas of the heart, sample points, non-rigid rotation and other geometry assumptions might evolve and allow for more refined computations prior to outflow.

Further, systole effectively yields some fluidic compression as well. A change in volume incurs a change in potential energy that is observed with or imputed by modern imaging techniques, and then reconciled with kinetic energy output.

For most non-symptomatic, healthy individuals, any such computation generally proves much lower than aortic outflow kinetic energy.

Inside the Chambers

In order to more precisely refine or confirm the total assessment of cyclic energy production, computations account for pumping contributions.

A cyclic pump, such as the heart, is considered a closed loop circuit. Diastole and systole continually repeat, including the flows they produce and their physiology. Physical, morphological, or physiological constraints are exploited, as cycles repeat.

Progressive “spiraling” depolarization from electrical stimulation of cardiac muscle culminates in systole, leading to fluidic motion. Forces inducing such motion include commonly known cardiac electrical stimulation waves and cardiac muscle contractions. Cardiac muscle chambers encircle blood, such that the enclosed fluid manifests electrical and magnetic fields.

Each heart chamber therefore invokes behavior of its enclosed fluid, effectively behaving as a coiled inductor.

Cardiac muscle encircles blood, where boundaries meet. Experiments demonstrate that blood is a better electrical conducting medium than muscle.

There is a relationship between electrical fields and magnetic fields. With atrial and/or ventricular muscle waves of voltage stimulation (e.g., via cellular action potential), the enclosed fluid reacts electrically and magnetically. Since ionic wave progress spatially via a spiraling configuration of muscle rather than all at once with mechanical wave compression occurring too, the boundary layer of fluid along chamber walls moves accordingly. Other fluid layers or internal streamline tubes of flow then incur motion.

Fluidic “current” in time follows input voltage. In other words, magnetic flux arises with current.

Current is equivalent to the flow of charge through any defined surface, such as fluid flow observed through a sample disk slice.

Boundary layer current as a function of cyclic time (i.e., current(t)) in a chamber is observed with modern imaging techniques and maximize velocity magnitude relative to internal fluid streamlines.

Current(t) induces magnetic flux, where total flux linked by a circuitry textbook inductor is:

λ(t)=L(t)*current(t) Equation 3

where L represents the inductance property of an inductor.

In general circuitry science, the rate at which an inductor's flux linkage builds up in time and is given by:

$$\frac{d λ ( t )}{d t} = voltage ( t ) \qquad Equation 4$$

For a linear inductor, magnetic flux builds up at a rate as voltage input, in time. Integrating both sides yields:

λ(t)=∫voltage(t)dt Equation 5

As measurable current(t) fluxes through a valve disk slice, unknowns are estimated at such cycle phase. For example, atrial current(t) through the mitral valve has associated flux already built up. According to general circuitry science, where inductance property L amounts to μ N² Area(t)/length(t):

$$λ ( t ) = ( \frac{μ {N}^{2} Area ( t )}{length ( t )} ) * current ( t ) \qquad Equation 6$$

Area(t) is the area of each fluidic coil slice and length(t) is fluidic length. These are observable and equivalent to their bounding constraints, e.g. left atrium plus mitral valve orifice.

N represents the number of coils, to be addressed by observable fluidic turns. In other words, it is the number of angular velocity spiral rotations in time relative to target spin axis, per given volume slice, N(t).

“μ” or “mu,” as used herein, represents the magnetic permeability of inductor's enclosed material, i.e., the magnetic permeability of the circulatory fluid. Magnetic permeability represents how permeable a material is to magnetization. For a biological organism, such fluidic core in all likelihood is not magnetically linear. Assuming a nonlinear magnetic fluidic core, sample points help ascertain boundary estimates or establish curves.

In example embodiments, fluidic μ varies by person, within a cycle, wave frequency, and more. Where oxygenated red blood cells (RBC) are involved, their diamagnetic and electrical behavior has been experimentally observed to be complex. In practice, this is approximated somehow by a μ(t) for an individual, if observable conditions support such an estimate.

Total flux(t) or λ(t) at that cycle phase has been built up by fluidic current tracking atrial P wave's ionization at boundary wall. Therefore μ at the time of mitral valve closure is sampled as:

$$μ ( {t}_{1} ) = \frac{\int voltage ( P wave ) d t * length ( t )}{{( N ( t ) )}^{2} * Area ( t ) * current ( {t}_{1} )} \qquad Equation 7$$

Wherein t₁ denotes t=mitral valve closure.

Flux state represents its past voltage history. In other words, flux state could serve as a state machine for cardiac cycles.

Therefore sampling magnetic permeability at a particular time μ(t₁), such as mitral valve closure, leads to state iteration through corresponding flux state μ(t₁).

$$\begin{matrix}λ ( {t}_{2} ) \approx {\int }_{{t}_{1}}^{{t}_{2}} voltage d t + {\int }_{- ∞}^{{t}_{1}} voltage d t \\ \approx {\int }_{{t}_{1}}^{{t}_{2}} voltage d t + λ ( {t}_{1} ) Equation 9\end{matrix} \qquad Equation 8$$

Further state iteration through λ(t) and sampled or imputed μ(t) refines current and energy estimations at cyclic time sample points.

For example, systolic estimations are compared to post-ejection current velocity estimates. For a meaningful efficiency comparison, using systolic mass estimation outflow, kinetic energy is further compared against systolic in-chamber energy production.

From a Given Engine State

If a given flux state λ(t) at t=mitral valve closure were equal for two different people, then their systolic energy production would not necessarily be equal. This is true even if their fluidic properties happened to be exactly the same.

Flux linkage builds up and chamber geometry affects how much, and therefore affects the magnetic field density which will contribute towards output magnitude.

Contributing to such inductance flux (aside from ranging μ(t) and current(t)), are from above:

$$( \frac{{( N ( t ) )}^{2} Area ( t )}{length ( t )} )$$

Another way to estimate N is from the enclosing chamber or using cardiac muscle fiber's effective spiral and/or coil density. If depolarization (i.e., activation) density distribution through such muscle (progressing cellular action potential) were to be smoothly distributed at boundary walls, then the number of turns N is effectively proportional to chamber length.

For a similar μ(t), or an assumed human curve, after term cancellation, flux builds up in proportion to N(t)=observable length(t)×area(t).

At the isovolumetric phase for example, ventricular geometry matters in flux buildup. All else being equal, the longer and wider the boundary interface the more buildup or the higher flux state can be achieved, even during torsion or perceived “wringing.”

This provides a quick way to assess potential systolic flux contribution.

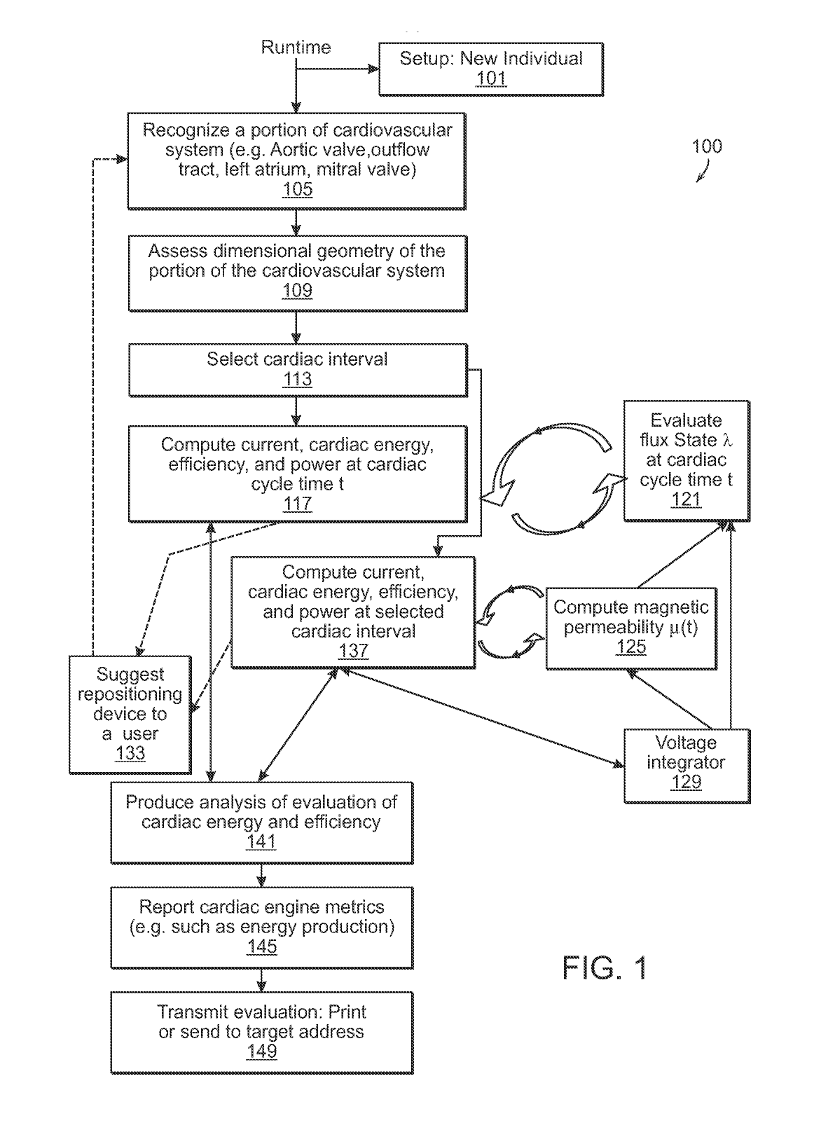

FIG. 1 illustrates a method for evaluating an individual's cardiovascular system according to the principles of the present invention. The method starts with setup 101 for a new individual. The application/computer system 100 automates recognition 105 of a portion of a cardiovascular system. The application/computer system 100 then assesses 109 the cardiovascular system in terms of its dimensional geometry. A cardiac interval is selected at step 113. Then, at step 117, system 100 computes cardiac current, energy, efficiency, and power at cyclic time t. Similarly, step 137 makes this calculation at the selected cardiac interval. If necessary, a repositioning of an imaging device is suggested to a technician 133. In the example embodiment shown in FIG. 1, flux state λ is evaluated 121 and magnetic permeability is computed 125. Voltage from, for example an EKG feed, is also tracked 129.

The computations yield an analysis of evaluation of cardiac energy and efficiency 141. Such metrics of the cardiac engine are reported 145 and transmitted to a target address 149.

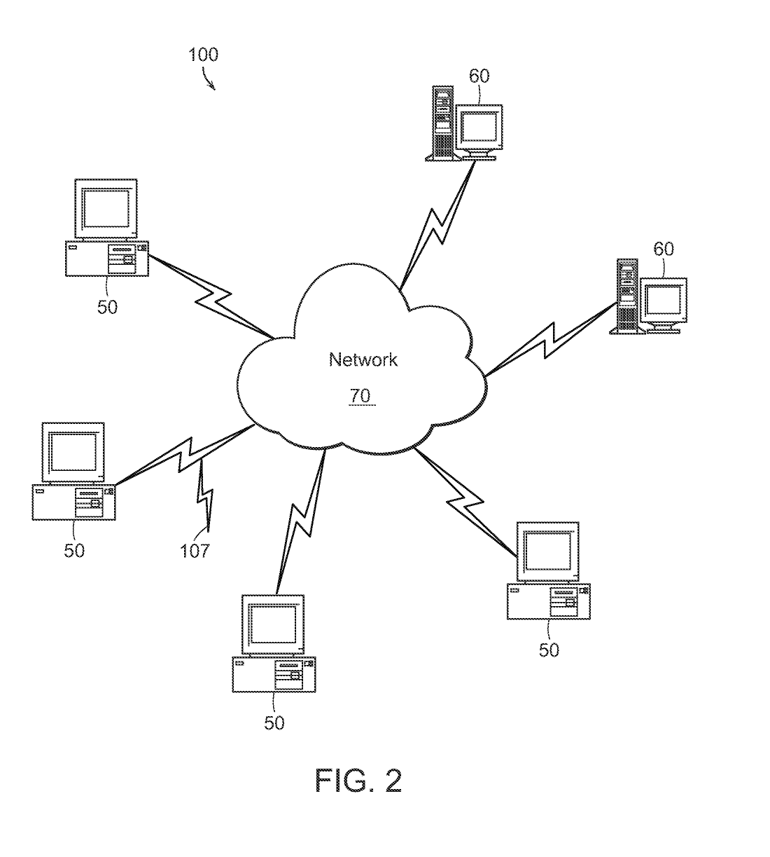

FIG. 2 illustrates a computer network or similar digital processing environment in which an embodiment of the present invention 100 may be implemented.

Client electronic devices 50 and server computers 60 provide processing, storage, and input/output devices executing application programs and the like. Client electronic devices 50 can also be linked through communications network 70 to other computing devices, including other client electronic devices/processes 50 and server computers 60. Electronic device 50 is any device including a processor, and can include a server, a computer, a laptop, a tablet, a smart phone, a cell phone and the like. In certain embodiments, electronic device 50 further includes one or more sensors or cameras. In certain embodiments, electronic device 50 connects to the Internet in order to upload data, an evaluative assessment, or the like to a health care provider, or alternately sends such information via email. Communications network 70 can be part of a remote access network, a global network (e.g., the Internet), a worldwide collection of computers, Local area or Wide area networks, and gateways that currently use respective protocols (TCP/IP, Bluetooth, etc.) to communicate with one another. Other electronic device/computer network architectures are suitable.

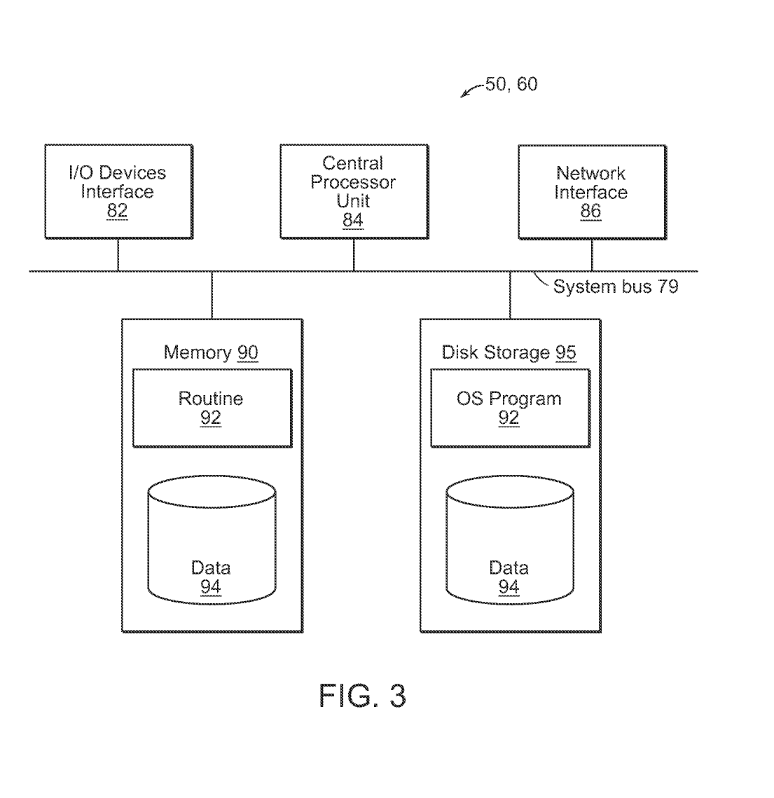

FIG. 3 is a diagram of the internal structure of a computer (e.g., client electronic devices 50 or server computers 60) in the computer system 100 of FIG. 2. Each electronic device 50 or server computer 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 2). Memory 90 provides volatile storage for computer software instructions 92 and data 94 used to implement an embodiment of the present invention (e.g., cardiac generative energy code 100 described and detailed above and in FIG. 1). Disk storage 95 provides non-volatile storage for computer software instructions 92 and data 94 used to implement an embodiment of the present invention. Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A computer system for evaluating an individual's cardiovascular system, the system comprising:
a recognition module configured to recognize a portion of an individual's cardiovascular system;
a geometry module coupled to the recognition module and configured to assess a dimensional geometry, physiology, or a combination thereof of the recognized portion of the individual's cardiovascular system at a cardiac cycle time t;
an interval selection module configured to select a cardiac cycle interval as a function of cardiac cycle time t;
an interval evaluation module responsive to the geometry module's assessment of the recognized portion of the individual's cardiovascular system and configured to compute current, cardiac power, cardiac energy, cardiac efficiency, or a combination thereof of the individual's cardiovascular system at the selected cardiac cycle interval, cardiac cycle time t, or a combination thereof, wherein the interval evaluation module indicates an evaluation of cardiac energy and cardiac efficiency;
a flux state module configured to evaluate inductive flux state λ of the individual's cardiovascular system at the cardiac cycle time t; and
a magnetic permeability module configured to compute magnetic permeability at the cardiac cycle time t;
wherein the inductive λ and μ are used to refine computation of current, cardiac power, cardiac energy, cardiac efficiency, or a combination thereof.

2. The computer system of claim 1, wherein the dimensional geometry assessed is area, volume, length, relative orientation, or a combination thereof of the recognized portion of the individual's cardiovascular system at the cardiac cycle time t.

3. The computer system of claim 1, further comprising a circulatory fluid property module configured to determine a permeability, a deformability, an oxygenation, or a combination thereof of a circulatory fluid.

4. The computer system of claim 1, further comprising a pulsatility module configured to determine a rotational velocity, a translational velocity, a volume, or a combination thereof by gauging one or more geometries and one or more velocities of a pulse wavefront and by gauging deformation of blood vessels.

5. The computer system of claim 1, further comprising a circulatory effect module configured to display a circulatory motion in one or more regions of the individual's cardiovascular system.

6. The computer system of claim 1, further comprising a boosting module configured to determine changes in vascular energy delivery.

7. The computer system of claim 1, further comprising a sensor module configured to connect one or more sensors.

8. The computer system of claim 1, further comprising a device module configured to connect one or more imaging devices.

9. The computer system of claim 8, wherein one of the one or more imaging devices is a camera.

10. The computer system of claim 8, further comprising a recommendation module configured to suggest repositioning of the one or more sensors or one or more imaging devices.

11. The computer system of claim 1, wherein the evaluation of cardiac energy and efficiency is an evaluation of incoming energy, output energy, kinetic energy, translational kinetic energy, rotational kinetic energy, or potential energy at the selected cardiac cycle interval or the cardiac cycle time t.

12. (canceled)

13. The computer system of claim 1, further comprising a cross-chambers module configured to determine differences in function between heart chambers.

14. A computer-based method of evaluating an individual's cardiovascular system, the method comprising:
a) in a recognition module, executable by a processor, recognizing a portion of an individual's cardiovascular system;
b) in a geometry module, executable by the processor, assessing a dimensional geometry, physiology, or a combination thereof of the recognized portion of the individual's cardiac structure at a cardiac cycle time t;
c) in an interval selection module, executable by the processor, selecting a cardiac cycle interval as a function of cardiac cycle time t;
d) in an interval evaluation module, executable by the processor, computing current, cardiac power, cardiac energy, efficiency, or a combination thereof at the selected cardiac cycle interval, cardiac cycle time t or a combination thereof to generate an evaluation of the individual's cardiovascular system, wherein the evaluation is an assessment of cardiac energy and efficiency;
e) in a flux state module, executable by the processor, evaluating inductive flux state λ of the individual's cardiovascular system; and
f) in a magnetic permeability module, executable by the processor, computing magnetic permeability μ at cardiac cycle time t; wherein the inductive λ and μ are used to refine computation of current, cardiac power, cardiac energy, cardiac efficiency, or a combination thereof.

15. The method of claim 14, wherein the dimensional geometry is area, volume, length, relative orientation, or a combination thereof at the cardiac cycle time t.

16. The method of claim 14, wherein the evaluation of cardiac energy and efficiency is an evaluation of incoming energy, output energy, kinetic energy, potential energy, translational kinetic energy or rotational kinetic energy at the selected cardiac cycle interval or the cardiac cycle time t.

17. (canceled)

18. The method of claim 14, further comprising:
suggesting repositioning of an imaging device;
producing an analysis of the evaluation of cardiac energy and efficiency; and
transmitting the individual's evaluation to a target address.

19. The method of claim 18, wherein producing the analysis of the evaluation of cardiac energy and efficiency comprises reporting of energy production; and further wherein the target address is a printer, an electronic mail recipient or a Cloud computing device.
